(19) United States
(12) Patent Application Publication
(10) Pub. No.: US 2021/0076646 A1
(43) Pub. Date: Mar. 18, 2021

(54) Aquarium Monitoring Device

(71) Applicant: Debra Cummings, Boynton Beach, FL (US)

(72) Inventor: Debra Cummings, Boynton Beach, FL (US)

(21) Appl. No.: 17/023,310

(22) Filed: Sep. 16, 2020

Related U.S. Application Data
(60) Provisional application No. 62/901,139, filed on Sep. 16, 2019.

(51) Int. Cl. A01K 63/00; G01N 33/18

(52) U.S. Cl. CPC A01K 63/006; G01N 33/18

(57) ABSTRACT

An intelligent aquarium management device that determines the appropriate temperature range and pH factor for particular species of fish in an aquarium. The aquarium owner first enters information describing the types of fish in the aquarium into the aquarium management device. The aquarium management device then determines the water temperature and pH factor of the aquarium. Once the temperature and pH factor information is determined, it is then compared with information in the database that lists the types of fish in the aquarium as well as the proper temperature range for each type of fish in the aquarium as well as the type of water (fresh/salt). If the aquarium management device determines that the aquarium temperature is in the correct range, it will indicate such. If the temperature is incompatible with the fish in the aquarium, an indication of that condition will also be output. Incompatibilities between species will also be determined, and the user will be notified.

### CROSS-REFERENCE TO RELATED APPLICATIONS

This application is related to, and claims the benefit of, the commonly owned co-pending provisional patent application entitled “Aquarium Monitoring Device”, filed Sep. 16, 2019, bearing U.S. Ser. No. 62/901,139 and naming Debra Cummings, the named inventor herein, as sole inventor, the contents of which is specifically incorporated by reference herein in its entirety.

### BACKGROUND OF THE INVENTION

### Technical Field

The present invention relates to aquariums. In particular, it relates to an intelligent device for managing aquariums in commercial and residential settings. The device is used to monitor both temperature and PH levels in aquarium water. In addition, it uses a database to store information and identify the number and types of fish in the aquarium. A database containing information about ideal temperature and PH levels for each type of fish in the aquarium is used to determine if there are any incompatible fish in the aquarium. An external software application is also available to provide detailed information to the aquarium user.

### Background

Today, many commercial and residential businesses maintain aquariums for decorative and/or hobby purposes. However, proper maintenance of aquariums requires a reasonable level of skill along with a significant level of diligence to properly care for the fish in the aquarium. If not properly maintained, the health of the fish will decline and perhaps the fish may perish due to poor maintenance and misunderstanding of species compatibility in the aquarium. In addition to concerns for the health of fish, the aquarium's owner will incur additional replacement costs due to improper care for the fish.

An important factor in maintaining an aquarium is proper regulation of water temperature. Different types of fish are healthiest at specific water temperature ranges which will vary from one species of fish to another. This presents an additional problem in that the aquarium owner may not be knowledgeable about the physical requirements of each of the various fish and their aquarium. It would be desirable to have a convenient method of determining the most desirable temperature range for particular type of fish in an aquarium.

Prior art temperature measurement devices typically require a probe to be placed in the water of the aquarium or alternatively securing a temperature measurement device to the external surface of the aquarium. Placement of the probe in the water as well as placing an external temperature measurement device on the outside surface of the aquarium each have drawbacks. The use of a probe is inconvenient and an external temperature measurement device creates an aesthetic problem. It would be desirable to have a method of measuring aquarium water temperature without using a probe, or detracting from the aquarium's appearance with an external temperature measurement device. It would be desirable to have a method of measuring water temperature from a distance, especially when transporting or changing water, thereby eliminating the need for inconvenient and unsightly prior art methods of temperature measurement. While the prior art has provided methods of monitoring aquariums, it has failed to provide a method of externally monitoring water temperature in aquariums.

Another important factor in aquarium management is maintaining the proper water ph level for the type of fish in the aquarium. Various species of fish can have their health adversely affected by water that has an incorrect PH level. It would be desirable to have a method of conveniently measuring PH level to ensure the safety of the fish.

### SUMMARY OF THE INVENTION

The present invention solves the foregoing problems by providing an intelligent aquarium management device. The aquarium owner first enters information describing the types of fish in the aquarium into the aquarium management device. The aquarium management device then determines the water temperature of the aquarium. Once the temperature is determined the intelligent aquarium management device then compares the measured temperature with information in the database that lists the types of fish in the aquarium as well as the proper temperature range for the type of fish in the aquarium. If the aquarium management device determines that the aquarium temperature is in the correct range, it will indicate such. Likewise, if the temperature is incompatible with the fish in the aquarium, an indication of that condition will also be output.

### BRIEF DESCRIPTION OF THE DRAWINGS

FIG. 1 illustrates a preferred embodiment of the intelligent aquarium management device communicating with an application on a conventional smart phone.

FIG. 2 illustrates a front view of a preferred embodiment of the display panel of the intelligent aquarium management device.

FIG. 3 illustrates a side view of a preferred embodiment of the intelligent aquarium management device.

FIG. 4 illustrates a preferred embodiment of the settings menu displayed of the intelligent aquarium management device.

FIG. 5 illustrates a rear view of a preferred embodiment of the intelligent aquarium management device, and showing the camera and the IRT sensor.

FIG. 6 illustrates a preferred embodiment of the tank data entry menu displayed of the intelligent aquarium management device.

FIG. 7 is a cross reference chart that shows the compatibility of one freshwater species of fish with different species of freshwater fish.

FIG. 8 is a cross reference chart that shows the compatibility of one saltwater species of fish with different species of saltwater fish.

FIG. 9 is a color chart off in which a user marks off the appropriate matching color after a water sample from the aquarium has tested by intelligent handheld device.

### DESCRIPTION OF THE PREFERRED EMBODIMENT

A general overview of the invention is as follows:

The preferred embodiment of the invention uses a self-contained intelligent handheld device. An alternative embodiment uses a handheld device in conjunction with a software application that can run on a smart phone, a computer, a laptop, etc. that communicates with the handheld device. Communication can be accomplished with any suitable means. In the preferred embodiment, Bluetooth is used as the preferred connection mains due to its ubiquitous presence on smart phones, laptops, etc. As a result, Bluetooth enables the software application to communicate with the handheld device with no additional cost for hardware infrastructure.

For ease of discussion, the specification uses the term “smart phone app” when describing the invention. However, the term “smart phone” as used herein is used to describe a variety of devices. For example, the smart phone can in fact be a conventional “smart” cellular phone, but it can also be a tablet computer, a notebook computer, a personal computer, etc. The only requirement is that it be an intelligent device that can communicate with the monitoring device. In addition, the term “fish” is used for convenience, but the term fish, was used herein includes other aquatic creatures, such as marine reptiles, turtles, jellyfish, etc. Also, in addition to commercial aquariums, the invention can also be used by commercial vehicles transporting fish from one location to another.

The device also allows the user to specify whether the aquarium uses salt water or fresh water. The type of water, stored by the system, can be used as a reminder when listing instructions for the aquarium owner when normal aquarium maintenance is being performed, such as when water is being added or replaced.

Since the device is portable, it can also be used by fishermen to gauge water temperature and ponds, lakes, etc., because water temperature may have an effect on fish behavior. In turn, fishermen can use the temperature information to estimate the likelihood that a fish may bite of fishermen's lure.

The invention provides Bluetooth-enabled handheld device that communicates with a software application. An advantage of using a software application is that that allows information related to specific fish species to be downloaded from the Internet or other sources thereby allowing the internal database in the handheld device to be easily updated when necessary, in addition, a software application can also store historical data related to ongoing temperature measurements, PH balance, and compatibility.

The present invention provides the ability to measure water temperature from outside the aquarium while giving an accurate reading of the water temperature inside the aquarium. This is possible because it has been found that infrared (IR) technology can be used for this purpose, providing that specific frequencies and steps are used.

When managing aquariums and may contain a wide variety of species using varying water types and temperatures, it is important to understand the critical optimal ranges of factors that may adversely affect the contents of the aquarium. For example:

Critical Optimal Ranges for Saltwater & Freshwater Aquariums

1. pH
a. Saltwater 8.0-8.3
b. Freshwater mixed 7.0-7.5
c. Freshwater cichlid 8.0-9.-
d. Freshwater discus 6.2-7.0

2. Alkalinity
a. Saltwater—3.5-4.5 mg/l 175-225 ppm
b. Freshwater mixed 1.2-2.0 mg/l 60-100 ppm
c. Freshwater cichlid 3.5-4.5 mg/l 175-225 ppm
d. Freshwater discus 0.61 mg/l 30-50 ppm
e. Freshwater depending fish species in tank

3. No3 Nitrate—0-40

4. No2—Nitrite—0-0.5

5. KH—Carbonate Hardness—80-120

6. GH General Hardness—30-120

7. Chlorine—0

8. Saltwater critical optimal range
a. Temperature 78-80
b. Calcium 380-450 ppm
c. Alkalinity 2.86-4.29 mg/l
d. Salinity 32-35 ppt2
e. pH 7.8-8.0
f. Magnesium Mg—1250-1400 ppm
g. Po4 phosphate—<0.03 ppm
h. Ammonia—NH3 0 ppm
i. Silica—Si—1 ppm
j. Iodine—0.06 ppm
k. No3 Nitrate—0-10 ppm
l. No2 Nitrite—0.0 ppm
m. Strontium Sr—7-9 ppm
n. ORP 250-400 mV
o. Boron B<10 ppm

Method 1: the Precise Color Spectrum Analyzer with Coded Classification Chart Conversion to APP System

An alternative way to determine the quality of the water is to place a sample of water from the aquarium into a vial. Then use the IRT device 28, and camera 29 to measure the color and obtain an image of the liquid in the vial. First, place the camera eye “o” directly to the location of the chosen object/vial, focus and snap. Then use the broad color spectrum data to classify color from a bandwidth of colors by separating components of light by their different degrees of refraction according to wavelengths from a scale between two extreme or opposite points. The results will output HEX #RGB—red green blue HSV—hue saturation value, HSL—hue saturation light, HSB—hue saturation brightness, CMYK—cyan magenta yellow black, and CIE LAB—CIE XYZ data from a full color spectrum. The color spectrum detector inside the camera device setting is accurate, vibrant and clearly analyzes the precise color with the backlight and pixelated vibrant display screen. For best results use white background. In the past traditional hard copy water chart comparison options were very controlled with a limited color scale and disintegrating clarity of color over time. The camera data will store, save and send the output detailed results giving the end user a precise accurate comparison report. All of the data will be saved and stored to a named tank data file to the app with user id, date and time—chemical water tests done for tank with results. Overall results will be stored and calculated for the wellbeing of all species in the named tank. 1) Log in FACE ID, 2) Choose preferred tank from tank list having data for each tank, 3) Scroll and choose TEST, 4) check camera icon inside chosen test 5) Test chosen will appear with color coded spectrum and data. The camera will open and match the exact color form spectrum data to object, 6) the camera will focus & store data to tank in camera setting focus mode calculate the color code from the (“0”) detector in focus mode color palette database installed from color spectrum coded charts (see aquarium charts) csv place “0” detector on image—or vial of chemical water sample or color strip or chart—for best results place behind a white background dominant wavelengths—UV, IR color temperature, luminance, RGB stored spectrum data and chart, —HSV-HSL-HSB-CMYK-CIE LAB CMYK spectrum test reader & data display reader will show: color coded data range spectrum for specified test camera, 7) SNAP (press button, color will focus and automatically find perfect match with exact reading, and 8) store when complete.

IR is normally used to read surface temperature. Normally, this results in the surface temperature of the outside of the aquarium being measured. However, IR can also be used to read either surface temperature and/or internal water temperature based on the IR frequency selected. When reading temperatures inside the aquarium, short wavelengths will pass through the glass wall of the aquarium at specific frequency wavelengths. In particular, IR frequency wavelengths at 0.7 to 0.4 microns can be used to read internal water temperature inside the aquarium. This is a significant improvement over the prior art in that the water temperature can be determined without contaminating the contents of the aquarium with a probe or detracting from the appearance of the aquarium by a permanently attached temperature gauge on the exterior of the aquarium.

Method 2: Precise RGB Color Spectrum Detecting System

The second way to resolve this issue is to match the color inside the vial with our irt device camera. Place the camera eye “O” directly to the location of the chosen object/vial, then focus and snap. The system uses a broad color spectrum data to classify color from a bandwidth of colors by separating components of light by their different degrees of refraction according to wavelengths from a scale between two extreme or opposite points the results will output HEX #-RGB-HSV-HSL-CMYK-CIE LAB—CIE XYZ data from a full color spectrum. The color spectrum detector inside the camera deviice setting is accurate, Vibrante and clearly analzyes the precise color with the backlight and pixelated vibrante display screen. In the past, traditional hard copy water chart comparison options were very controlled with a limited color scale and disintegrating clarity of color over time. The camera data will store, save and send the output detailed results giving the end user a precise accurate comparison report. All data will save and store to named tank and to app with user id, date and time. Overall results will calculate for the wellbeing of all species in the named tank.

First in camera setting select focus mode calculate the color code from the (“O”) detector in focus mode color palette database CSV place “O” detector on image—or vial of chemical water sample. For the best results place behind a white background display reader will show: range spectrum dominant wavelengths UV˜IR luminance RGB-HSV-HSL-CMYK-CIE LAB when complete save to clipboard and app output display will provide color name %-HEX #-RGB-HSV-HSL-CMYK-CIE LAB-CIE XYZ beneficial to end user: stored color image, color data, username, time and date.

APP Settings for Database
1. Enter data options.
2. Hard copy transfer through camera keyboard from laptop, tablet, smartphone, etc., app.

Start
1. Every entry will automatically store user id, date and time
2. Login—identify username, time, and date.

Set Water
1. Select and store water preference.
2. User must logout-save to app-log back in to change water preference

Menu
1. Temperature only—temperature tank
2. Water testing and maintenance: color chart conversion or color spectrum detector camera
3. Camera—transfer chemical data to app—detect color

Menu
1. Create tank name
2. Create basic profile
3. Create new profile and data
4. New plant profile
5. New coral profile
6. New invertebrate profile
7. New reef profile
8. New decorative objects
9. Camera
10. Maintenance: water testing and maintenance

Application Data
1. Data is continuously updated and generated stored in tank profile and to application.

Temperature Only
1. Generate data of input and output temperature in display panel.
2. Enter and store data for username, date, time, and temperature.

Create Tank
1. Create name and identify tank type and size of tank, lighting, filters, etc.
2. Choose which chemical water tests to be stored and taken for this tank
3. Set reminders to calendar
4. Save and store—lock or unlocked with face id

Create Basic Profile
1. Choose from fish species and/or add new
2. Choose from plant species and/or add new
3. Choose from coral and/or add new
4. Choose from invertebrates and/or add new
5. Choose from plants and/or add new
6. Choose from reef and/or add new
7. Choose from decorative object and/or add new

Basic Profile
1. Enter species or scroll down from data
2. Generate data for optimal temperature for that species
3. Generate data for compatable species
4. Enter save and store data for species to specified tank

Create New Species Detailed Profile
1. Choose from fish species and/or add new
2. Choose from plant species and/or add new
3. Choose from coral and/or add new
4. Choose from invertebrates and/or add new
5. Choose from plants and/or add new
6. Choose from reef and/or add new
7. Choose from decorative object and/or add new

Create New Species Detailed Profile
1. Identify species enter or scroll down from data
2. generate data for optimal temperature for that species
3. Generate data for compatible species
4. Create name
5. Take photo of species
6. Enter new photo of species
7. Identify and enter special characteristics
8. Identify and enter temperament
9. Where purchased
10. Price
11. Select hierarchy of species
12. Generate data for life expectancy
13. Compatibility and danger data for new species in profile
14. Receive data for compatibility and danger of other species in current tank
15. Temperature
16. Receive data for optimal temperature of species
17. Receive data for optimal temperature of overall species in current tank
18. Set new data for optimal temperature range in current tank
19. Save and store data to specified tank

Use the same procedure for creating profiles for new plant. coral, invertebrates, Reefs, etc. Username date, time, and will be automatically save and stored after every use. Water maintanence, color testing options, color chart conversion, color spectrum detector nitrate (no3), nitrite (no2), hardness (gh), chlorine (mg/l), alkalinity (kh) ph balance, ph hard, ammonia, calcium, phosphate salinity, iodine, magnesium, strontium 1) rinse test vial air dry 2) sample water to fill line with dropper place small amount of aquarium water exactly to line on vial 3) take chemical test dropper, drop one drop of chemical into vial of aquarium water 4) mix 5) develop color 6) choose color chart to app or color spectrum detector from device for accurate results data generate data receive data for top 10 suggested compatible species receive data of important traits receive data for overall diet compatibility receive data for overall supplements compatibility, receive data for overall plant compatibility receive data for overall reef compatibility receive data for overall coral compatibility receive data for overall invertebrates compatibility receive data for overall placement compatibility receive data for overall water flow compatibility receive data for overall lighting compatibility, receive data for suggested new compatible species, enter and add new species data to specified tanks add and generate new data generate data for added species generate list of alive identified species generate list of dead identified species generate data for overall optimal temperature range for existing species and plants/coral/reef/inverts generate data for overall water testing results for existing species and plants/coral/reef/inverts generate data for added species compatibility or danger to existing fish and/or plants *set new optimal temperature for overall species plants reef inverts coral specified tanks lowest—highest range save and store optional overall data and print options receive data for top 10 suggested compatible species receive data of important traits receive data for overall species compatibility receive data for overall temperature setting receive data for overall food compatibility receive data for overall plant compatibility, receive data for overall water test results, receive data for suggested new compatible species optional overall costs receive data for overall costs generated fish species receive data for overall costs generated plants reef coral inverts receive data for overall costs generated decorations receive data for overall costs generated for food receive total data for overall generated costs, store temperature data, username, date and time, choose temperature & species, add chemical water tests to chosen tank.

The database used by the invention includes data related to the type of water in the aquarium. Saltwater and freshwater fish can only survive in one type of water. As a result, fish related data is categorized by the type of water they live in.

The output of the aquarium management device can be audio, visual display, indicator lights, etc. Likewise, for convenience of the user, of device can be switched by the user to indicate temperatures and both the Fahrenheit and/or Celsius temperatures scales. In addition, the preferred embodiment of invention allows the user to select the language used to communicate with the user.

In addition to the foregoing discussion of temperature management, the device also includes an additional important feature in that it can also measure the PH factor of the water. To safely care for fish in an aquarium, knowing the water temperature is only one part of the solution. An improper PH factor can be as hazardous to fish as water that is too hot or work too cold. The invention also solves this problem by providing a compartment in the device into which a small sample of the water is placed. The compartment includes a PH tester that determines the PH level of water. When the water tested, both the temperature and the PH factor a report to the user. If the device determines that either value is incorrect it is reported to the user. Likewise, the user is also notified when both the temperature and/or PH factor for the type of fish and the aquarium is within proper range.

The invention also makes use of a database in which the user enters information about each specific fish in the aquarium. The information may quote the type of fish, where it was purchased, when it was the added to the aquarium, the name of the vendor from which it was purchased, and even a photographic image of the actual fish, or a stock image of that type of fish. The user can also add any other information that the user deems appropriate.

The software application used by the invention can analyze the database for a variety of purposes. For example, it can determine whether different types of fish are incompatible with one another, either due to incompatible temperature or PH ranges, or to aggressive behavior that once species of fish may exhibit towards another. Likewise, a software application may look for problems such as fish purchased from one vendor that becomes sick and die shortly after purchase vs. fish purchased from another vendor that have long and healthy lives. This will save the user expenses by informing the user as to which vendors do not provide a quality product.

One preferred embodiment of the invention allows the user to enter data related to the aquarium and the fish residing therein are paper data sheet that can be scanned and entered into the software application. Alternatively, they can also paid directly entered into the software application through a data entry screen on the device. The device then uploads the data correctly and for the software application via its Bluetooth or other suitable connection.

In addition to storing data related to the fish in the database, the database can also be used to store information related to other items within the aquarium. For example, aquariums typically contain a variety of plants for decorative purposes. The application also stores information regarding plants used in aquariums, and compares that information with the types of fish and the aquarium to determine if there's an incompatibility between the types of fish and the plants in its environment.

The physical format of the aquarium monitoring device can take any suitable form. While it can be implemented at a small form factor such as one in which it resembles a conventional pen, a small form factor will naturally limit the size of a display screen. A slightly larger form factor, such as something the size of an ordinary remote control for a television, will enable a larger displays that can display information in a richer format. Alternatively, the information gathered by the aquarium monitoring device can be transferred to an application in a smart phone that will allow a superior output format using color, audio, video, etc. For example, color can be used to indicate when the water temperature is in the proper temperature range or pH balance.

In addition to outputting information related to the aquarium, the aquarium monitoring device can also output information related to its own status, such as battery status, etc.

An optional feature of invention is this an integral camera in the aquarium monitoring device, along with a run for whole telephone connection. This allows an individual using the software application in their cellphone to dial the home phone number and access live video showing the condition of the aquarium.

The aquarium monitoring device can be fabricated from any suitable natural, such as carbon fiber, aluminum, plastic, etc. Likewise, a replaceable battery or a chargeable battery. When using a rechargeable battery, the preferred embodiment envisions using a USB Port to charge the battery.

The invention provides several benefits. It saves money for the aquarium owner by enhancing the health of the fish in the aquarium. Of course, it also helps the fish by keeping them in the healthiest possible environment. In addition, an inexperienced aquarium owner is also helped because they may not know the optimum temperature or pH factor range for a given fish, may not know if there are incompatibilities between different types of fish, or may not know how to maintain records related to the aquarium. They're aquarium monitoring device, in conjunction with the software application provides an effective way for both experienced users and to novice users to manage their aquarium, and to provide superior care for the fish within the aquarium.

Having discussed the invention in general, we turn now to a discussion of the figures.

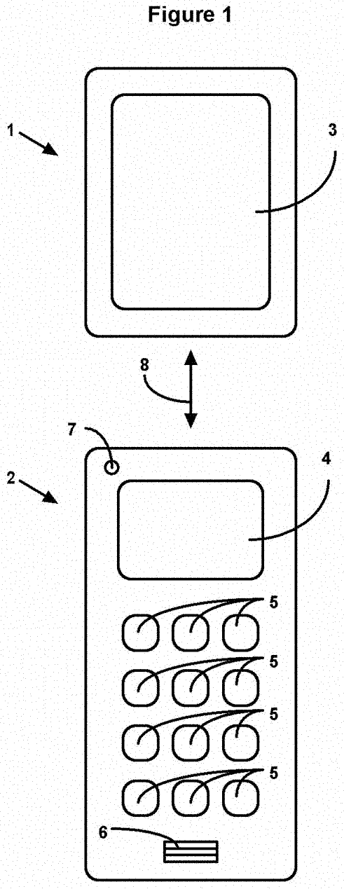

FIG. 1 illustrates a preferred embodiment of the intelligent aquarium management device 1 communicating with an application on a conventional smart pone 2. As shown in this figure, the intelligent aquarium management device 1 has a display screen 3 for use when interacting with a user.

This figure also illustrates a conventional smart phone 2 that communicates with the intelligent aquarium management device 1. The standard features of a typical smart phone 1, the display screen 4, the data entry keys 5, the microphone 6, and the camera 7 are also shown.

In the preferred embodiment, the invention uses Bluetooth technology do to its ubiquitous use throughout the electronics industry as well as its low cost and convenience. However, those skilled in the art will recognize that any other suitable technology can be used, not only wireless technology, but even wired technology such as a USB cable interface.

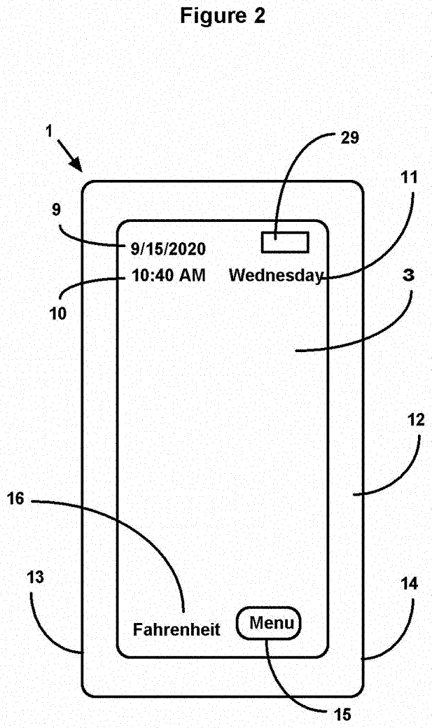

FIG. 2 illustrates a front view of a preferred embodiment of the display panel of the intelligent aquarium management device 1. This figure illustrates the data displayed on the initial startup screen when the intelligent aquarium management device 1 is turned on. In the preferred embodiment, The date 9, the time of day 10, the day of the week 11, the battery status 29, and the temperature scale 16 are displayed. Also shown in this figure Is the menu button 15 which is used to access other menus and features of the invention.

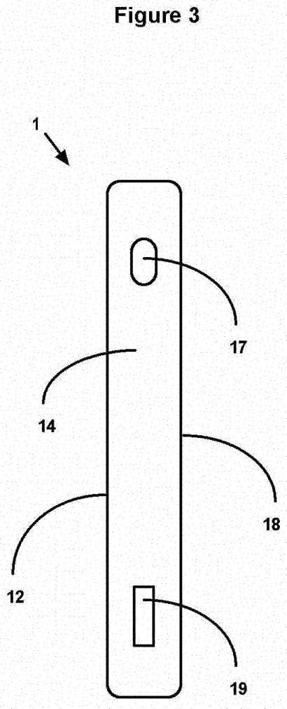

FIG. 3 illustrates a side view of a preferred embodiment of the intelligent aquarium management device 1. This view illustrates the power button 17, and a USB Port 19. The front surface 12, the rear surface 18, and the side edge 14 are also shown. Of course, the power button 17 and the USB Port 19 can be placed in any convenient location.

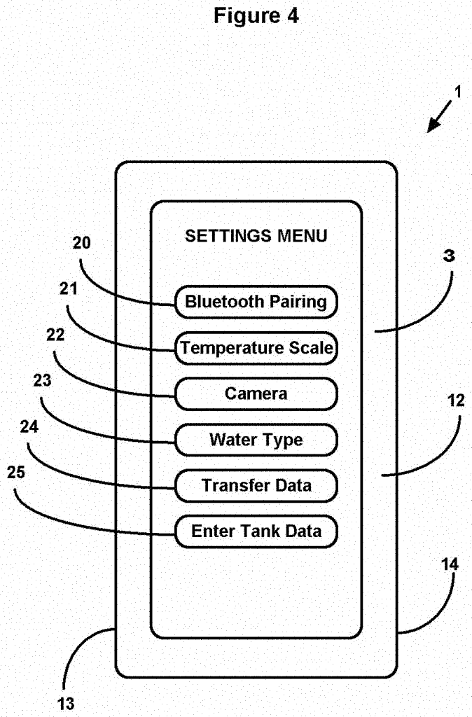

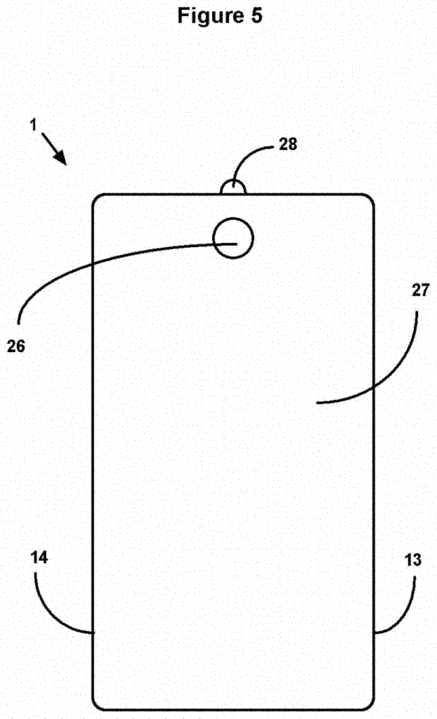

FIG. 4 illustrates a preferred embodiment of the settings menu 15 displayed of the intelligent aquarium management device 1. When this menu is accessed, it allows the user to select sub-menus by selecting any of the categories of information 20-25 that the user may want to enter, or actions that the user may want to take. For example, the user would select the Bluetooth button 20 to connect with the smart phone 2. The user would select the temperature scale button 21 to display measurements in either the Fahrenheit or Celsius scale. The camera button 22 would be used to activate the camera 26 (Shown in FIG. 5). The water type (fresh water or salt water) would be selected by the water type button 23. The transfer data button 24 would be selected when the user decides to transfer data created by actions taken when any of the other buttons are selected. The tank data button 25 allows the user to enter data related to the aquarium and the contents thereof, including fish, water type, plants, coral, ornaments, etc.

FIG. 5 illustrates a rear view of a preferred embodiment of the intelligent aquarium management device 1, and showing the camera 26 and the IRT sensor 28.

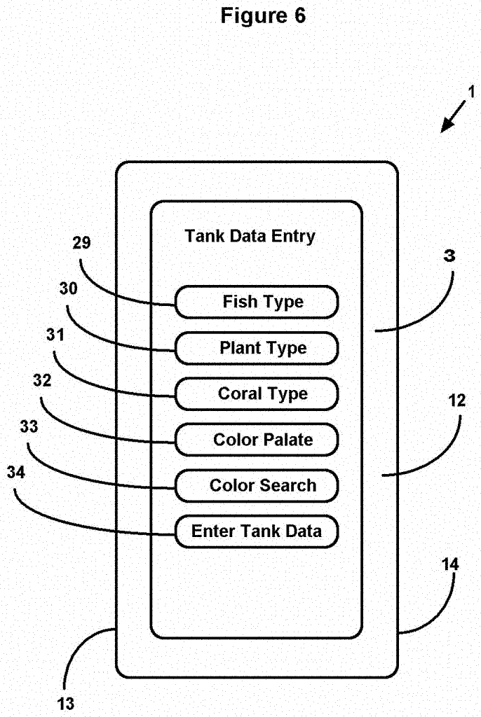

FIG. 6 illustrates a preferred embodiment of the tank data entry menu displayed of the intelligent aquarium management device 1. The tank data entry menu allows the user to select the type of data they want to enter Enter the application in the smart phone 2.

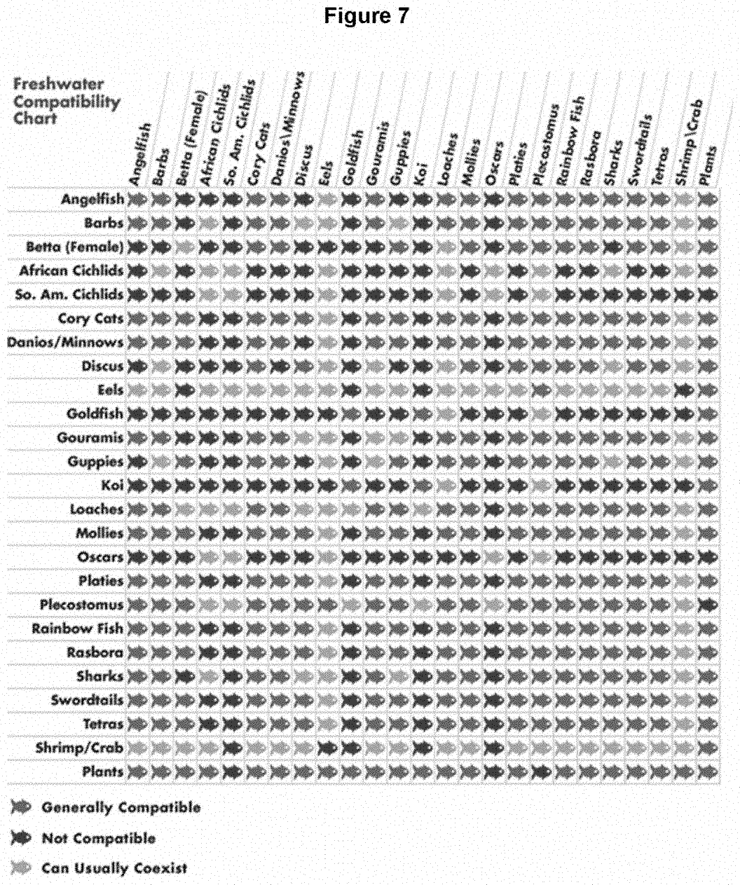

FIG. 7 is a cross reference chart that shows the compatibility of each freshwater species fish with different species of freshwater fish.

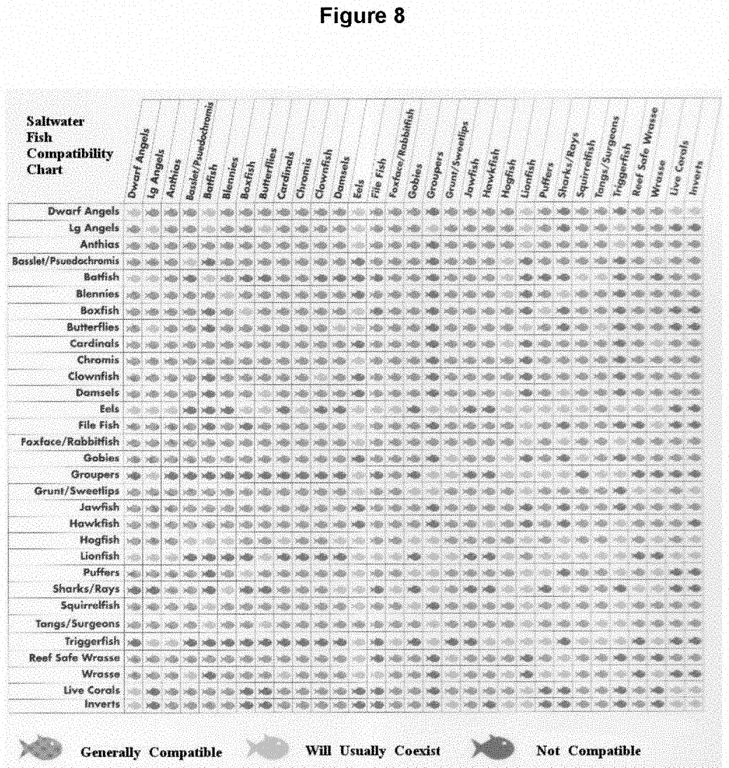

FIG. 8 is a cross reference chart that shows the compatibility of each saltwater fish species with different species of saltwater fish.

FIG. 7-8 are useful to anyone who maintains an aquarium, but they are especially useful to novice users who may inadvertently place two species of fish together who do not coexist peacefully.

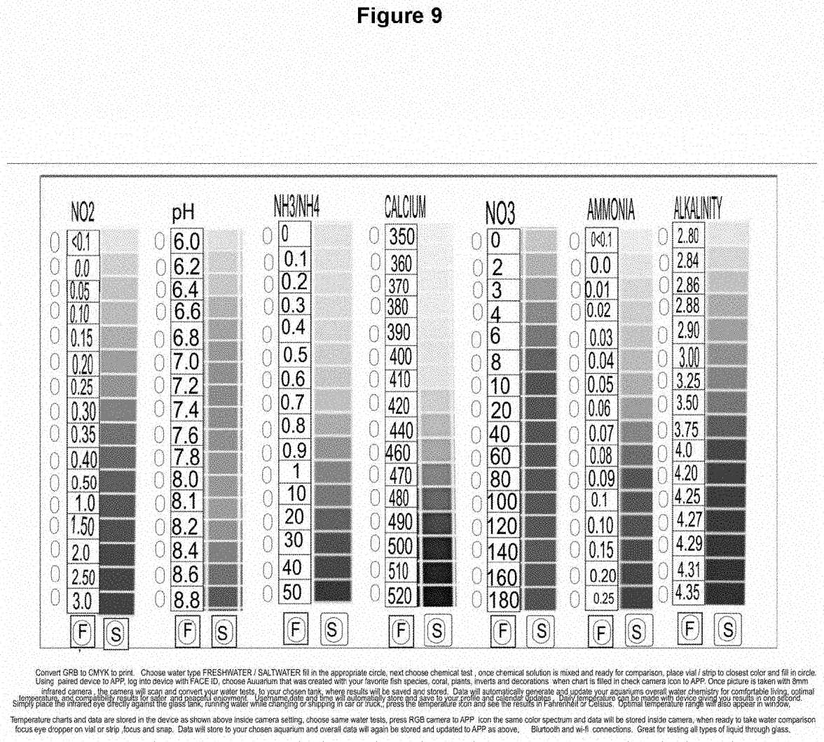

FIG. 9 is a color chart form in which a user marks off the appropriate matching color after the IRT 28 tests the water in the aquarium is tested by the intelligent handheld device 1. The user then marks the appropriate matching box on the color chart form is then photographed by the camera 26 which then uploads the data to the application in the smart phone 2. The use of the color form allows the user to correctly identify the appropriate color which may be distorted when IRT data is translated to other data formats. This eliminates inadvertent errors that may occur do to data conversion.

While the invention has been described with respect to a preferred embodiment thereof, it will be understood by those skilled in the art that various changes in detail may be made therein without departing from the spirit, scope, and teaching of the invention. For example, the number and type of fish, the type of water (i.e., salt or fresh water), the communications technology can vary from hard wired to wireless, the materials used to fabricate the aquarium monitoring device, etc., can all vary. Accordingly, the invention herein disclosed is to be limited only as specified in the claims. I claim:

What is claimed is:

1. An aquarium monitoring system, comprising:
a temperature measuring device for measuring aquarium temperature and water PH;
a Bluetooth communications interface in the temperature measuring device;
a smart phone having a database application that includes information related to types of fish and compatibility in the aquarium, and the appropriate temperature range for each specific type of fish, the smart phone also uses a camera to scan data input via paper forms;
a Bluetooth communications interface in the smart phone for communicating with the Bluetooth communications interface in the proper temperature measurement, pH, identification and species compatibility device.
